(19) United States
(12) Patent Application Publication
(10) Pub. No.: US 2018/0360382 A1
(43) Pub. Date: Dec. 20, 2018

(54) OPTICAL MEASUREMENT DEVICE WITH PRESSURE FEEDBACK FUNCTION

(71) Applicant: PixArt Imaging Inc., Hsin-Chu City (TW)

(72) Inventor: Cheng-Nan Tsai, Hsin-Chu City (TW)

(21) Appl. No.: 15/626,166

(22) Filed: Jun. 18, 2017

(51) Int. Cl. A61B 5/00; A61B 5/145; A61B 5/1455

(52) U.S. Cl. CPC A61B 5/6843; A61B 5/14546; A61B 2562/0242; A61B 5/681; A61B 2562/0238; A61B 5/14551

(57) ABSTRACT

An optical measurement device with pressure feedback function includes an optical detecting module and a stretchable connective belt. The optical detecting module is adapted to output at least one optical detecting signal to detect pressure applied on an object. The stretchable connective belt is assembled with the optical detecting module and utilized to tie the optical detecting module on the object, and the stretchable connective belt is deformed to vary the pressure generated by the optical detecting module.

### BACKGROUND OF THE INVENTION

### 1. Field of the Invention

The present invention relates to an optical measurement device, and more particularly, to an optical measurement device with pressure feedback function.

### 2. Description of the Prior Art

A smart wearable device is utilized to put on user's wrist, and the optical detecting module of the smart wearable device projects an optical detecting signal onto the wrist skin to acquire blood vessel information for health examination. The conventional smart wearable device has a non-elastic watchstrap, the wrist skin is deformed by pressure of the smart wearable device while the non-elastic watchstrap is tied on the user's wrist, the blood vessel information may be interfered because the blood vessel is squelched, and biological detection accuracy of the smart wearable device is incorrect and unstable accordingly.

### SUMMARY OF THE INVENTION

The present invention provides an optical measurement device with pressure feedback function for solving above drawbacks.

According to the claimed invention, an optical measurement device with pressure feedback function includes an optical detecting module and a stretchable connective belt. The optical detecting module is adapted to output at least one optical detecting signal to detect pressure applied on an object. The stretchable connective belt is assembled with the optical detecting module and utilized to tie the optical detecting module on the object, and the stretchable connective belt is deformed to vary the pressure generated by the optical detecting module. The stretchable connective belt is extended to reduce the said pressure for increasing biological detection accuracy of the optical detecting module. The stretchable connective belt includes a length adjusting mechanism, and the length adjusting mechanism is activated to adjust an encircling length of the stretchable connective belt in accordance with the pressure detected by the optical detecting module.

According to the claimed invention, the optical detecting module computes signal intensity about an optical reflecting signal generated from the object, and compares the signal intensity with a threshold to determine whether the pressure is set within tolerance. The signal intensity is a ratio of an alternating current to a direct current about the optical reflecting signal. The optical detecting module projects two optical detecting signals with different wavelengths onto the object, and determines a variation of the pressure in accordance with optical reflecting signals generated from the object. The two optical detecting signals are respectively projected onto different layers having individual depths inside the object.

According to the claimed invention, the stretchable connective belt can include a resilient belt portion, and a plurality of marks is separately formed on the resilient belt portion for identifying a length variation of the resilient belt portion. The stretchable connective belt further can include a non-resilient belt portion overlapped above the resilient belt portion, an edge of the non-resilient belt portion is fixed on the resilient belt portion, and the other edge of the non-resilient belt portion is movable relative to the resilient belt portion. The stretchable connective belt further can include a resilient belt portion and a non-resilient belt portion connected side by side, and a plurality of marks is separately formed on the resilient belt portion.

According to the claimed invention, stretchable connective belt can include a belt portion and an elastic component, the elastic component is connected between the belt portion and the optical detecting module, the belt portion is extended via the elastic component in accordance with the pressure. The elastic component is a torsional spring rotatably disposed on the optical detecting module via an axle, and the belt portion is rolled up by the torsional spring.

The wearable optical measurement device of the present invention disposes the stretchable connective belt on the optical detecting module, the stretchable connective belt utilizes extension of the resilient belt portion or auto-rolling function of the elastic component to release its strain and to accordingly decrease the pressure applied on the object by the optical detecting module, then the optical detecting module can determine whether quantity of the said strain and the said pressure conforms to a predetermined demand by comparing the signal intensity with the threshold, and remind the user to keep or adjust the encircling length of the stretchable connective belt for the preferred biological detection accuracy of the optical detecting module.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of an optical measurement device with pressure feedback function according to a first embodiment of the present invention.

FIG. 2 is a diagram of the optical measurement device in different operation processes according to the first embodiment of the present invention.

FIG. 3 is a diagram of the optical measurement device according to a second embodiment of the present invention.

FIG. 4 is a waveform diagram of signal intensity about the optical reflecting signal according to an embodiment of the present invention.

FIG. 5 and FIG. 6 are waveform diagrams of the signal intensity about optical reflecting signals according to another embodiment of the present invention.

FIG. 7 is a diagram of the optical measurement device in different operation processes according to a third embodiment of the present invention.

FIG. 8 is a diagram of the optical measurement device according to a fourth embodiment of the present invention.

### DETAILED DESCRIPTION

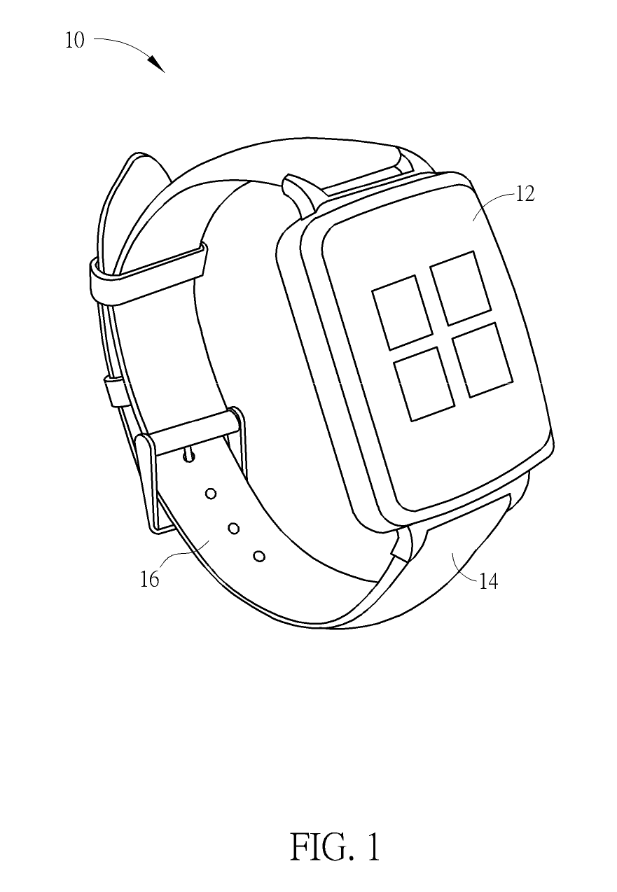

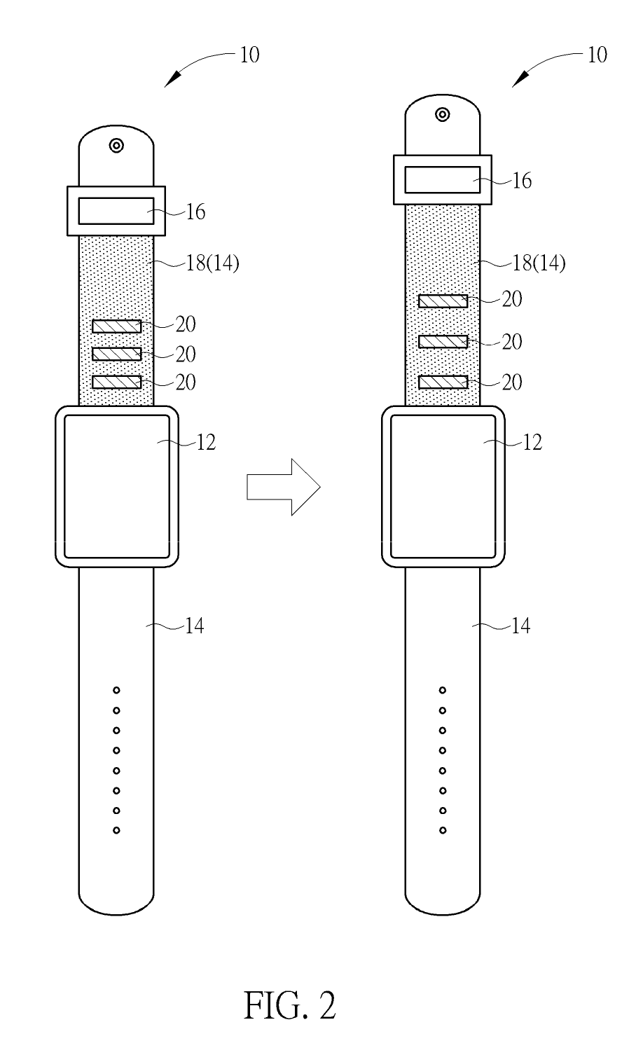

Please refer to FIG. 1 and FIG. 2. FIG. 1 is a diagram of an optical measurement device 10 with pressure feedback function according to a first embodiment of the present invention. FIG. 2 is a diagram of the optical measurement device 10 indifferent operation processes according to the first embodiment of the present invention. The optical measurement device 10 can include an optical detecting module 12 and a stretchable connective belt 14. The stretchable connective belt 14 is assembled with the optical detecting module 12 to tie the optical detecting module 12 on an object, and the object can be the wrist, the ankle, the neck, or any limbs of a user. The stretchable connective belt 14 may have two belts and a length adjusting mechanism 16, the two belts are respectively connected with opposite edges of the optical detecting module 12, and the length adjusting mechanism 16 can be a retainer ring structure utilized to link the two belts and to adjust an encircling length of the stretchable connective belt 14 while the optical measurement device 10 binds through the stretchable connective belt 14.

The optical detecting module 12 can be fastened on the wrist by the stretchable connective belt 14, and the optical detecting module 12 is used to output at least one optical detecting signal to detect vessel information about the wrist, such like hemoglobin oxygen saturation. Even though the encircling length is able to be adjusted via the length adjusting mechanism 16, a blood capillary of the surface layer may still be squashed by pressure of the optical detecting module 12 and the optical detecting module 12 acquires incorrect vessel information, so that the stretchable connective belt 14 is accordingly deformed and extended to reduce the pressure applied on the object for preferred biological detection accuracy of the optical detecting module 12. The foresaid pressure further can be represented as strain of the stretchable connective belt 14

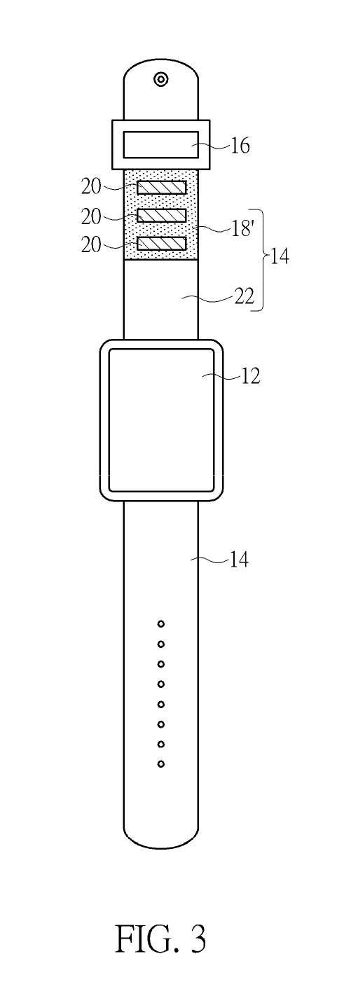

In the first embodiment, the stretchable connective belt 14 further has a resilient belt portion 18 connected with the optical detecting module 12, and a plurality of marks 20 is formed on the resilient belt portion 18 separately. An interval between the adjacent marks 20 is enlarged while the resilient belt portion 18 is extended, so as to identify a length variation of the resilient belt portion 18 and the related encircling length of the stretchable connective belt 14. In addition, the stretchable connective belt 14 can be designed as having a resilient belt portion 18′ and a non-resilient belt portion 22 connected side by side. Please refer to FIG. 3. FIG. 3 is a diagram of the optical measurement device 10 according to a second embodiment of the present invention. In the second embodiment, the resilient belt portion 18′ is deformed and the non-resilient belt portion 22 is constant while the stretchable connective belt 14 is extended. The plurality of marks 20 are formed on the resilient belt portion 18′ for identifying the length variation of the stretchable connective belt 14.

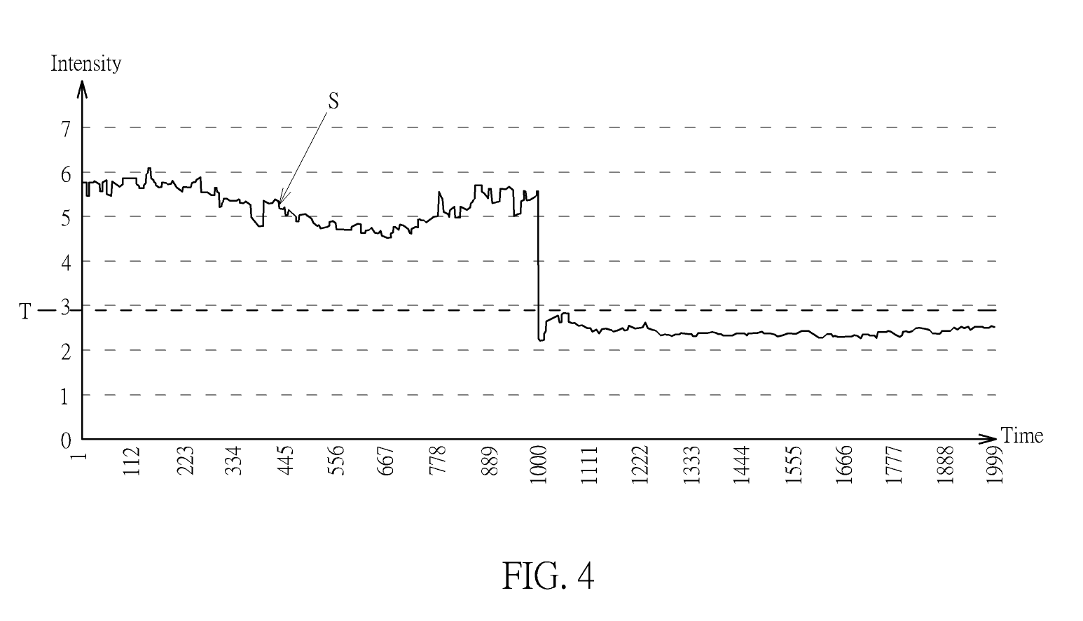

While the pressure applied on the object by the optical detecting module 12 is released via deformation of the stretchable connective belt 14, the optical detecting module 12 receives an optical reflecting signal generated from the object for accurate computation. Please refer to FIG. 4. FIG. 4 is a waveform diagram of signal intensity about the optical reflecting signal according to an embodiment of the present invention. The optical detecting module 12 computes the signal intensity S about the optical reflecting signal, the signal intensity S may drop off while the pressure is greater than tolerance. As shown in FIG. 4, the stretchable connective belt 14 can be gradually tightened by the user, the signal intensity S is slightly varied for some time and then suddenly descended while the pressure is out of the tolerance. Therefore, the optical detecting module 12 continuously compares the signal intensity S with a threshold T, the optical detecting module 12 keeps quiet as the signal intensity S conforms to the threshold T (such like being larger than the threshold T) and can output a warning as the signal intensity S does not conform to the threshold T (such like being lower than the threshold T), so the user may use the length adjusting mechanism 16 to vary the encircling length.

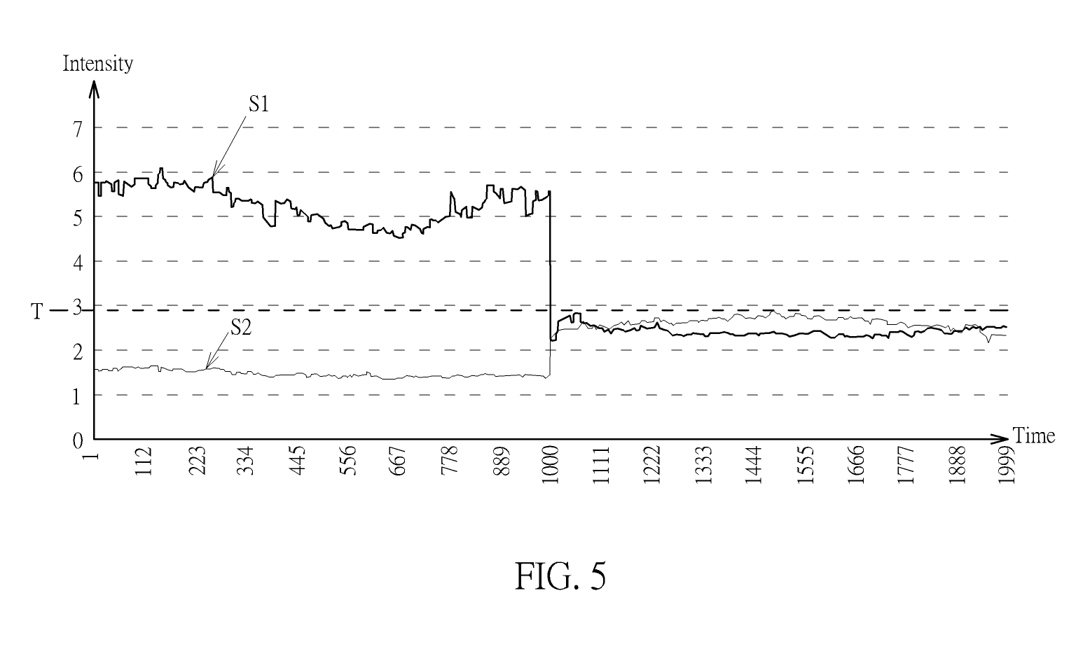

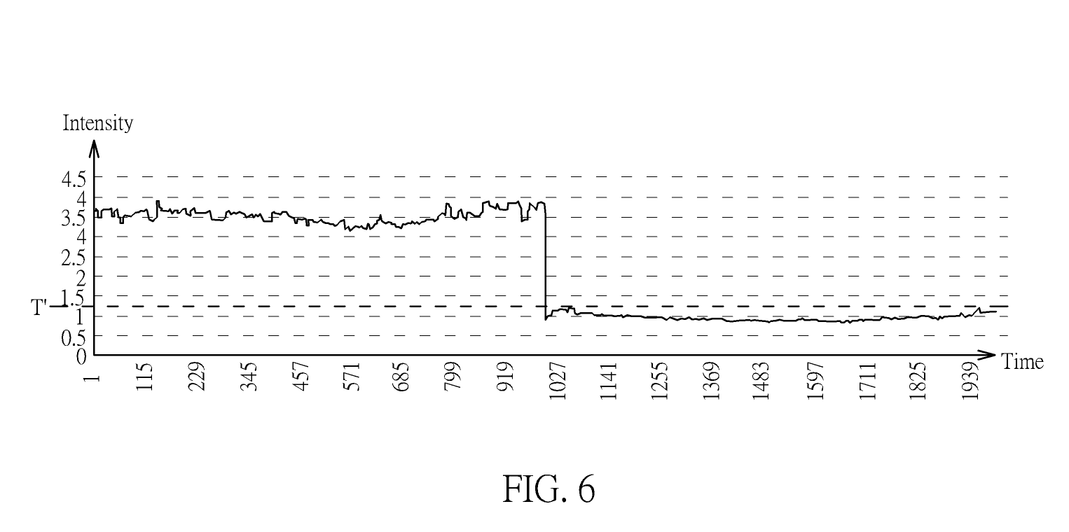

Please refer to FIG. 5 and FIG. 6. FIG. 5 and FIG. 6 are waveform diagrams of the signal intensity about optical reflecting signals according to another embodiment of the present invention. In this embodiment, the optical detecting module 12 projects two optical detecting signals with different wavelengths onto the object, receives two optical reflecting signals and acquires the signal intensity S1 and S2 accordingly. The two optical detecting signals are respectively projected onto different layers having individual depths inside the object, the signal intensity S1 (which is transformed by the optical reflecting signal with a short wavelength) can be descended and the signal intensity S2 (which is transformed by the optical reflecting signal with a long wavelength) can be ascended while the pressure is out of the tolerance, and a ratio of the signal intensity S1 to the signal intensity S2 is computed and utilized to compare with the threshold T′. The threshold T′ may be transformed from or identical with the threshold T.

When the object is not squashed or squash of the object is under acceptance, the said ratio conforms to the threshold T′ and is varied slightly, and the optical detecting module 12 can provide preferred biological detection accuracy; when the said ratio does not conform to the threshold T′, the optical detecting module 12 can find out the abnormal detection and output the warning to remind an unacceptable variation of the pressure applied on the object. It should be mentioned that the signal intensity S, S1 and S2 can be a perfusion index and be a ratio of an alternating current to a direct current about the optical reflecting signal; however, definition of the signal intensity is not limited to the foresaid statement, which depends on design demand.

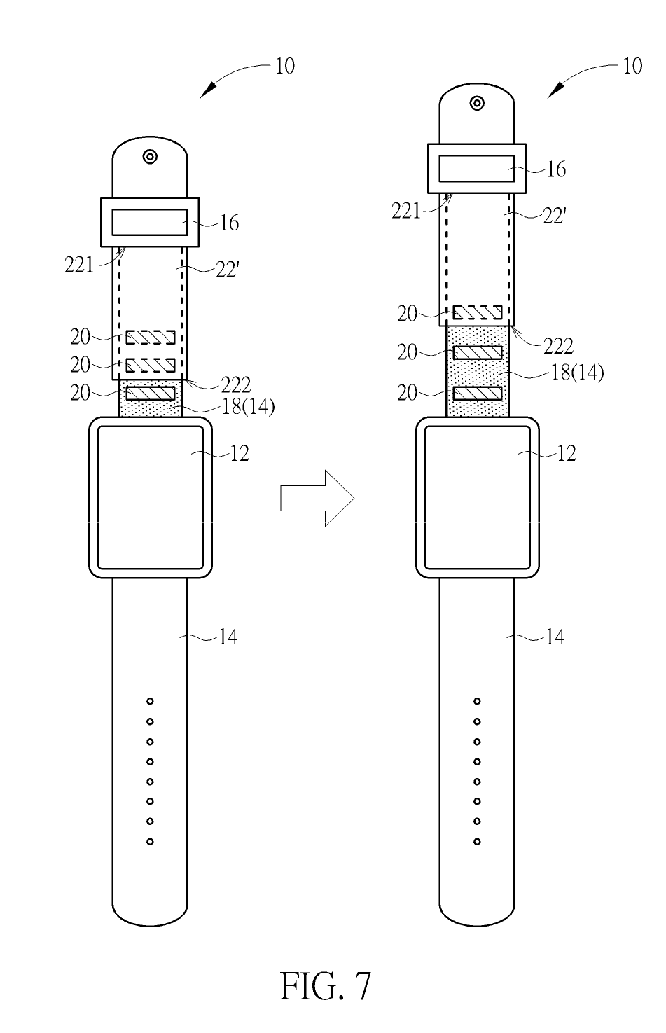

Please refer to FIG. 7. FIG. 7 is a diagram of the optical measurement device 10 in different operation processes according to a third embodiment of the present invention. The stretchable connective belt 14 can include the resilient belt portion 18 and a non-resilient belt portion 22′, the resilient belt portion 18 is connected between the length adjusting mechanism 16 and the optical detecting module 12, and the non-resilient belt portion 22′ is partly overlapped above the resilient belt portion 18. An edge 221 of the non-resilient belt portion 22′ is fixed on the resilient belt portion 18, and the other edge 222 of the non-resilient belt portion 22′ is unconstrained and can be movable relative to the resilient belt portion 18. The user can identify the length variation of the resilient belt portion 18 via indication of the edge 222.

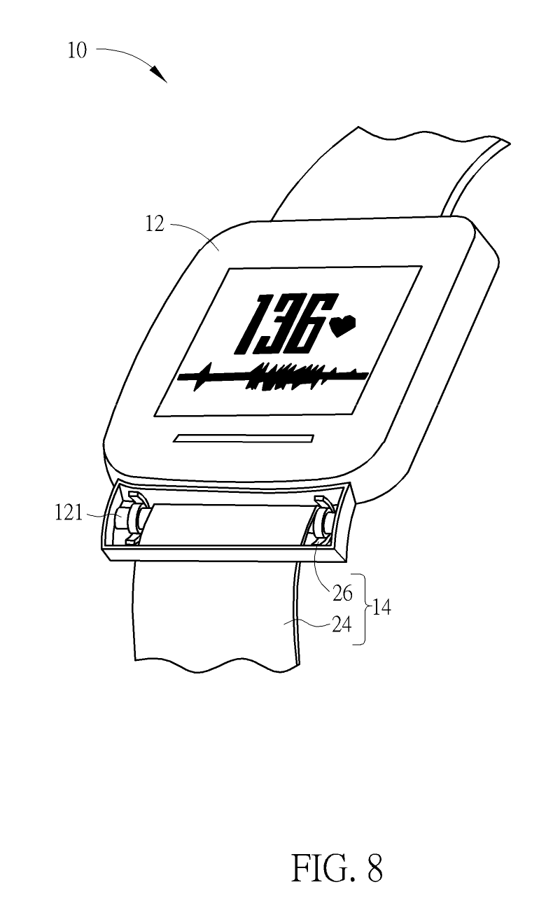

Please refer to FIG. 8. FIG. 8 is a diagram of the optical measurement device 10 according to a fourth embodiment of the present invention. The stretchable connective belt 14 may include a belt portion 24 and an elastic component 26. The elastic component 26 is a torsional spring rotatably disposed on an axle 121 inside the optical detecting module 12, and connected between the belt portion 24 and the optical detecting module 12. The belt portion 24 is rolled up by the elastic component 26 and can be extended via rotation of the elastic component 26 in accordance with the pressure applied on the object. For example, the belt portion 24 can be pulled out while the pressure is increased, and the elastic component 26 is compressed to store an elastic recovering force accordingly; as the stretchable connective belt 14 is unwound, the belt portion 24 is rolled up by the elastic recovering force to suitably tie the optical detecting module 12 on the object. That is, the belt portion 24 can be a resilient belt or a non-resilient belt.

In conclusion, the wearable optical measurement device of the present invention disposes the stretchable connective belt on the optical detecting module, the stretchable connective belt utilizes extension of the resilient belt portion or auto-rolling function of the elastic component to release its strain and to accordingly decrease the pressure applied on the object by the optical detecting module, then the optical detecting module can determine whether quantity of the said strain and the said pressure conforms to a predetermined demand by comparing the signal intensity with the threshold, and remind the user to keep or adjust the encircling length of the stretchable connective belt for the preferred biological detection accuracy of the optical detecting module.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An optical measurement device with pressure feedback function, comprising:
an optical detecting module adapted to output at least one optical detecting signal to detect pressure applied on an object; and
a stretchable connective belt assembled with the optical detecting module and utilized to tie the optical detecting module on the object, the stretchable connective belt being deformed to vary the pressure generated by the optical detecting module.

2. The optical measurement device of claim 1, wherein the stretchable connective belt is extended to reduce the said pressure for increasing biological detection accuracy of the optical detecting module.

3. The optical measurement device of claim 1, wherein the stretchable connective belt comprises a length adjusting mechanism, the length adjusting mechanism is activated to adjust an encircling length of the stretchable connective belt in accordance with the pressure detected by the optical detecting module.

4. The optical measurement device of claim 1, wherein the optical detecting module computes signal intensity about an optical reflecting signal generated from the object, and compares the signal intensity with a threshold to determine whether the pressure is set within tolerance.

5. The optical measurement device of claim 4, wherein the signal intensity is a ratio of an alternating current to a direct current about the optical reflecting signal.

6. The optical measurement device of claim 1, wherein the optical detecting module projects two optical detecting signals with different wavelengths onto the object, and determines a variation of the pressure in accordance with optical reflecting signals generated from the object.

7. The optical measurement device of claim 6, wherein the two optical detecting signals are respectively projected onto different layers having individual depths inside the object.

8. The optical measurement device of claim 1, wherein the stretchable connective belt comprises a resilient belt portion, and a plurality of marks is separately formed on the resilient belt portion for identifying a length variation of the resilient belt portion.

9. The optical measurement device of claim 8, wherein the stretchable connective belt further comprises a non-resilient belt portion overlapped above the resilient belt portion, an edge of the non-resilient belt portion is fixed on the resilient belt portion, and the other edge of the non-resilient belt portion is movable relative to the resilient belt portion.

10. The optical measurement device of claim 1, wherein the stretchable connective belt comprises a resilient belt portion and a non-resilient belt portion connected side by side, and a plurality of marks is separately formed on the resilient belt portion.

11. The optical measurement device of claim 1, wherein the stretchable connective belt comprises a belt portion and an elastic component, the elastic component is connected between the belt portion and the optical detecting module, the belt portion is extended via the elastic component in accordance with the pressure.

12. The optical measurement device of claim 11, wherein the elastic component is a torsional spring rotatably disposed on the optical detecting module via an axle, and the belt portion is rolled up by the torsional spring.
